(19) United States
(12) Patent Application Publication
(10) Pub. No.: US 2024/0350518 A1
(43) Pub. Date: Oct. 24, 2024

(54) COMPOSITIONS FOR TREATMENT OF HAIR LOSS

(71) Applicant: INNOCAN PHARMA LTD, Herzliya (IL)

(72) Inventor: Nir AVRAM, Metar (IL)

(73) Assignee: Innocan Pharma Ltd., Herzliya (IL)

(21) Appl. No.: 18/574,774

(22) PCT Filed: Jul. 26, 2022
(86) PCT No.: PCT/IL2022/050802

§ 371 Date: Dec. 28, 2023

Related U.S. Application Data
(60) Provisional application No. 63/226,185, filed on Jul. 28, 2021.

(51) Int. Cl. A61K 31/00; A61K 31/522; A61P 17/14

(52) U.S. Cl. CPC A61K 31/658; A61K 31/522; A61P 17/14

(57) ABSTRACT

Described herein are compositions which can be used to treat and prevent hair loss. The compositions comprise a cannabinoid, preferably cannabidiol. The compositions may comprise other ingredients that act synergistically with the cannabinoid to enhance hair growth, increase hair strength, or promote hair thickness in patients in need thereof. Further described are methods for treatment of hair loss comprising administering such compositions.

### CROSS REFERENCE TO RELATED APPLICATIONS

Benefit is claimed to U.S. Provisional Patent Application No. 63/226,185 filed Jul. 28, 2021; the contents of which are incorporated by reference herein in its entirety.

### FIELD

Provided herein are compositions comprising cannabinoids for treatment of hair loss.

### BACKGROUND

Hair loss, otherwise known as alopecia, can affect men and women on scalps or other parts of the body. Scalp hair loss is part of the natural aging process in men, but may also be contributed to by various factors such as medical conditions, medications, or hormonal changes.

Early hair loss may negatively impact an individual's self-image. Propensity for hair loss is often hereditary. However, medical intervention should be sought if hair loss does not follow standard patterns or occurs at an early age.

### SUMMARY

Described herein are compositions which can be used to treat and prevent hair loss. The compositions comprise a cannabinoid, preferably cannabidiol.

The foregoing and other objects, features, and advantages will become more apparent from the following detailed description.

### BRIEF DESCRIPTION OF THE DESCRIBED SEQUENCES

The nucleic and/or amino acid sequences provided herewith are shown using standard letter abbreviations for nucleotide bases, and one letter code for amino acids, as defined in with 37 CFR 1.831 through 37 CFR 1.835. Only one strand of each nucleic acid sequence is shown, but the complementary strand is understood as included by any reference to the displayed strand. The Sequence Listing is submitted as an XML file named INNO 3200-9-2, approximately 3 KB, created Jul. 24, 2022.

SEQ ID NO: 01 is the amino acid sequence of Myristoyl Pentapeptide-17 polypeptide.

SEQ ID NO: 02 is the amino acid sequence of Octapeptide-2 polypeptide.

### DETAILED DESCRIPTION

Unless otherwise noted, technical terms are used according to conventional usage. Definitions of common terms in pharmaceutical sciences can be found in Troy et al. Remington: The Science and Practice of Pharmacy. Published by Lippincott Williams & Wilkins, 2006. In case of conflict, the present specification, including explanations of terms, will control. In addition, all the materials, methods, and examples are illustrative and not intended to be limiting.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia and is used herein to indicate a non-limiting example. Thus, the abbreviation “e.g.” is synonymous with the term “for example.”

### Definitions

Cannabinoid: A cannabinoid is a chemical compound that acts on cannabinoid receptors in cells in mammals, including in humans. Cannabinoids can be manufactured synthetically or obtained from various parts of the genus Cannabis, in particular, from the species Cannabis sativa. Cannabinoids from the Cannabis plant are referred to as phytocannabinoids. Two preferred cannabinoids according to various embodiments, are (−)-trans-Δ⁹-tetrahydrocannabinol, and/or isomers thereof (THC) and cannabidiol (CBD). Alternatively, a cannabinoid may be in the form of Cannabis extract. Alternatively, a cannabinoid may be in the form of a synthetic cannabinoid. Compositions described herein may comprise one cannabinoid or multiple cannabinoids, such as a combination of CBD and THC.

### Description of Embodiments

Described herein, according to some embodiments, are compositions comprising a cannabinoid, for topical administration, for use in treating hair loss.

Compositions described herein may be formulated as a emulsion, gel, cream, lotion, spray, eye liner, mascara, hair oil, or hair serum.

The amount of cannabinoid may range from between 0.001% by weight to 1% by weight, preferably between 0.1% to 1% by weight, most preferably 0.5% by weight.

A single cannabinoid or a mixture of cannabinoids, such as a Cannabis extract, comprising multiple cannabinoids may be used in compositions described herein. Optionally, the cannabinoid is CBD. Alternatively, the cannabinoid is THC, CBN, or CBG.

According to preferred embodiments, the compositions comprise a cannabinoid and an additional active agent. The additional active agent may be caffeine. Caffeine is a phosphodiesterase inhibitor which has been shown to stimulate cell proliferation and stimulate hair follicle growth. The amount of caffeine present in the composition is preferably between 0.1% and 1% by weight, optionally 0.5% by weight.

According to an embodiment the additional active agent may be an agent that stimulates, promotes, strengthens or maintains hair growth. Optionally, the agent may be bimatoprost. Optionally, the agent may be minoxidil. Optionally, the additional active agent may be: ginseng extract, Biotinoyl Tripeptide-1, Myristoyl Pentapeptide-17, Octapeptide-2, hydrolyzed wheat protein, arginine, Arctium majus Root Extract, Chondrus crispus Extract, Ocimum basilicum Hairy Root Culture Extract, Pisum sativum (Pea) Sprout Extract, Capsicum frutescens Fruit Extract, Milk protein, Diaminopyrimidine Oxide Oxothiazolidinecarboxylate, or Lindera strychnifolia Root Extract. Optionally, the agent may be an agent that has a vasodilating effect.

The amount of the additional active agent may be between 0.0001% and 10% by weight, optionally between 0.01% by weight and 5% by weight.

Similarly, according to an embodiment, the compositions may comprise two or more additional active agents.

Described herein, according to some embodiments, are methods for treatment or prevention of hair loss using a cannabinoid, applied via the topical route to the skin of a patient in need thereof. Optionally, the cannabinoid is CBD. Alternatively, the cannabinoid is THC, CBN, or CBG.

Administration of the compositions described herein may be performed topically, by applying to the skin, scalp, or area where hair regrowth is desired. The composition should be applied to the on a daily basis, optionally, twice, three of four times daily. Preferably, the composition, in the form of an emulsion, gel, cream, lotion, solution, spray, eye liner, mascara, hair oil, or hair serum is applied and left on the area affected with hair loss for at least one hour. Optionally, the composition is applied before bed and left on overnight.

The amount administered per application for a composition may vary from between

0.1 g to 30 g per administration.

Cannabinoids, in particular CBD, may have a synergistic effect in the treatment of hair loss when used in combination with an additional agent.

Optionally, the composition may be applied in combination with microneedling. Microneedling is a process in which a skin roller with small needles is rolled over an affected area, for example, an area of the scalp having hair loss, and the needles cause minor skin injuries. The compositions described above may be administered subsequent to microneedling.

An embodiment of the invention relates to a packaged pharmaceutical composition for treatment of hair loss, wherein the composition comprises a cannabinoid and at least one additional active agent, and the package is selected from the group consisting of: a spray bottle, a bottle with a dropper, or a bottle having a nozzle, or a roll-on device wherein the rolling element is equipped with microneedles to simultaneously stimulate skin with needles while applying the composition.

Described herein, according to an embodiment, is a composition for treatment of a patient suffering from hair loss, or a method for treatment of a patient suffering from hair loss comprising administering to the patient a composition comprising a cannabinoid, applied via the topical route to the skin or scalp of a patient in need thereof. Optionally, the composition further comprises at least one additional active agent. Optionally, the at least one additional active agent is selected from the group consisting of: caffeine, minoxidil, bimatoprost, ginseng extract, Biotinoyl Tripeptide-1, Myristoyl Pentapeptide-17, Octapeptide-2, hydrolyzed wheat protein, arginine, Arctium majus Root Extract, Chondrus crispus Extract, Ocimum basilicum Hairy Root Culture Extract, Pisum sativum (Pea) Sprout Extract, Capsicum frutescens Fruit Extract, Milk protein, Diaminopyrimidine Oxide Oxothiazolidinecarboxylate, apigenin, oleanoic acid and Lindera strychnifolia Root Extract. Optionally, the composition is in the form of an emulsion, gel, cream, lotion, spray, eye liner, mascara, hair oil, or hair serum. Optionally, the cannabinoid is present in the composition in an amount between 0.001% and 5% by weight. Optionally, the cannabinoid is CBD. Optionally, the CBD is present in an amount of 0.5% by weight of the composition. Optionally, the composition is left on the skin or scalp of the patient for at least one hour. Optionally, the composition is applied in combination with microneedling. Optionally, the amount administered of the composition per administration is between 0.1 g and 30 g. Optionally, the additional active agent is present in the composition in an amount of between 0.0001% by weight and 5% by weight. Optionally, an additional agent is caffeine.

The following examples are provided to illustrate certain particular features and/or embodiments. These examples should not be construed to limit the disclosure to the particular features or embodiments described.

### EXAMPLES

### Example 1

A lotion for treatment of or prevention of hair loss and having CBD is prepared using the ingredients in table 1:

TABLE 1

| | Amount (weight/ | |
|---|---|---|
| Ingredient | weight) % | Function |
| Water | 65.80 | Solvent/Carrier |
| Caffeine | 0.50 | Stimulation of |
| | | hair growth |
| Ethanol | 25.00 | Solvent |
| Hemp Oil having 3% CBD | 1.00 | Active ingredient |
| Butylene glycol | 2.00 | |
| Phenoxyethanol 90.0% | 1.00 | Preservative |
| Ethylhexylglycerin 10.0% | | |
| PPG-26-Buteth-26 (and) | 1.00 | Solubilizer |
| PEG-40 Hydrogenated | | |
| Castor Oil (and) Water | | |
| Fragrance | 0.20 | Fragrance |
| Biotinoyl Tripeptide-1 | 3.00 | Active |
| containing composition | | |
| Ginseng extract | 0.50 | Promotes hair growth |

The biotinoyl tripeptide-1-containing composition in this and in the following examples is a commercially available composition, and comprises the following: Butylene Glycol-Water (Aqua), PPG-26-Buteth-26, PEG-40 Hydrogenated Castor Oil, Apigenin, Oleanolic Acid, and Biotinoyl Tripeptide-1, having the sequence Biotin-Gly-His-Lys. The concentration of biotinoyl tripeptide-1 in the Biotinoyl Tripeptide-1 containing composition is 200 ppm when 3.00% of biotinoyl tripeptide-1-containing composition is added. The Biotinoyl Tripeptide-1 containing composition further comprises 400-550 ppm Apigenin and 250-350 ppm Oleanolic acid.

In a main container, water and caffeine are heated to 75° C. and stirred until dissolved, then cooled while stirring. In another container, hemp oil is dissolved in ethanol at 45° C. and added to the main container. In another container, butylene glycol is combined with preservative and added to the main container. In another container, the solubilizer is mixed with the perfume and added while mixing to the main container. At 40° C. Biotinoyl Tripeptide-1 containing composition and ginseng extract are added to the main container. The composition's pH may adjusted by adding small amounts of citric Acid or sodium hydroxide 20% aqueous solutions

### Example 2

A lotion for treatment of or prevention of hair loss and having CBD is prepared using the ingredients in table 2:

TABLE 2

| | Amount | |
|---|---|---|
| Ingredient | (weight/weight) % | Function |
| Water | 65.80 | Solvent/Carrier |
| Caffeine | 0.50 | Stimulation of |
| | | hair growth |
| Ethanol | 25.00 | Solvent |
| CBD (Isolate, 97%) | 0.50 | Active |
| | | ingredient |
| Hemp Oil | 0.50 | Carrier |
| Butylene glycol | 2.00 | Humectant |
| Phenoxyethanol 90.0% | 1.00 | Preservative |
| Ethylhexylglycerin 10.0% | | |
| PPG-26-Buteth-26 (and) | 1.00 | Solubilizer |
| PEG-40 Hydrogenated | | |
| Castor Oil (and) Water | | |
| Fragrance | 0.20 | Fragrance |
| Biotinoyl Tripeptide-1 | 3.00 | Active |
| containing composition | | |
| Ginseng extract | 0.50 | Promotes |
| | | hair growth |

The composition is prepared as in example 1, with the added step of adding CBD to the hemp oil in ethanol at 45° C.

### Example 3

A lotion for treatment of or prevention of hair loss and having CBD is prepared using the ingredients in table 3:

TABLE 3

| | Amount | |
|---|---|---|
| Ingredient | (weight/weight) % | Function |
| Water | 65.70 | Solvent/Carrier |
| Caffeine | 0.50 | Stimulation of |
| | | hair growth |
| Ethanol | 25.00 | Solvent |
| CBD (Isolate, 97%) | 0.50 | Active |
| | | ingredient |
| Hemp Oil | 0.50 | Carrier |
| Butylene glycol | 2.00 | Humectant |
| Phenoxyethanol 90.0% | 1.00 | Preservative |
| Ethylhexylglycerin 10.0% | | |
| PPG-26-Buteth-26 (and) | 1.00 | Solubilizer |
| PEG-40 Hydrogenated | | |
| Castor Oil (and) Water | | |
| Fragrance | 0.20 | Fragrance |
| Biotinoyl Tripeptide-1 | 3.00 | Active |
| containing composition | | |
| Ginseng extract | 0.50 | Promotes |
| | | hair growth |
| Biotin | 0.10 | Hair growth |
| | | stimulating |
| | | agent |

The composition is prepared as in example 2, with the added step of adding biotin with the ginseng extract to the main container at 40° C.

### Example 4

A lotion for treatment of or prevention of hair loss and having CBD is prepared using the ingredients in table 4:

TABLE 4

| | Amount | |
|---|---|---|
| Ingredient | (weight/weight) % | Function |
| Water | 65.65 | Solvent/Carrier |
| Caffeine | 0.50 | Stimulation of |
| | | hair growth |
| Ethanol | 25.00 | Solvent |
| CBD (Isolate, 97%) | 0.50 | Active |
| | | ingredient |
| Hemp Oil | 0.50 | Carrier |
| Butylene glycol | 2.00 | Humectant |
| Phenoxyethanol 90.0% | 1.00 | Preservative |
| Ethylhexylglycerin 10.0% | | |
| PPG-26-Buteth-26 (and) | 1.00 | Solubilizer |
| PEG-40 Hydrogenated | | |
| Castor Oil (and) Water | | |
| Fragrance | 0.20 | Fragrance |
| Biotinoyl Tripeptide-1 | 3.00 | Active |
| containing composition | | |
| Ginseng extract | 0.50 | Promotes |
| | | hair growth |
| Biotin | 0.10 | Hair growth |
| | | stimulating |
| | | agent |
| Myristoyl Pentapeptide-17 | 0.05 | Active |

Myristoyl Pentapeptide-17 is an agent having the structure Myr-Lys-Leu-Ala-Lys-Lys-NH2 (SEQ ID NO 01) when Myr represents a myristoyl residue.

The lotion is made as described in Example 3, with the addition of myristoyl pentapeptide-17 to the main container at 40° C.

### Example 5

A lotion for treatment of or prevention of hair loss and having CBD is prepared using the ingredients in table 5:

TABLE 5

| | Amount | |
|---|---|---|
| Ingredient | (weight/weight) % | Function |
| Water | 65.65 | Solvent/Carrier |
| Caffeine | 0.50 | Stimulation of |
| | | hair growth |
| Ethanol | 25.00 | Solvent |
| CBD (Isolate, 97%) | 0.50 | Active |
| | | ingredient |
| Hemp Oil | 0.50 | Carrier |
| Butylene glycol | 2.00 | Humectant |
| Phenoxyethanol 90.0% | 1.00 | Preservative |
| Ethylhexylglycerin 10.0% | | |
| PPG-26-Buteth-26 (and) | 1.00 | Solubilizer |
| PEG-40 Hydrogenated | | |
| Castor Oil (and) Water | | |
| Fragrance | 0.20 | Fragrance |
| Biotinoyl Tripeptide-1 | 3.00 | Active |
| containing composition | | |
| Ginseng extract | 0.50 | Promotes |
| | | hair growth |
| Biotin | 0.10 | Hair growth |
| | | stimulating |
| | | agent |
| Octapeptide-2 | 0.05 | Active |

The lotion is made as described in Example 3, with the addition of Octapeptide-2 (acetyl octapeptide-2) to the main container at 40° C. The sequence of Octapeptide-2 is Glu-Gln-Leu-Lys-Thr (SEQ ID NO 02).

### Example 6

A lotion for treatment of or prevention of hair loss and having CBD is prepared using the ingredients in table 6:

TABLE 6

| | Amount | |
|---|---|---|
| Ingredient | (weight/weight) % | Function |
| Water | Up to 100% | Solvent/Carrier |
| Caffeine | 0.50 | Stimulation of |
| | | hair growth |
| Ethanol | 25.00 | Solvent |
| CBD (Isolate, 97%) | 0.50 | Active |
| | | ingredient |
| Hemp Oil | 0.50 | Carrier |
| Butylene glycol | 2.00 | Humectant |
| Phenoxyethanol 90.0% | 1.00 | Preservative |
| Ethylhexylglycerin 10.0% | | |
| PPG-26-Buteth-26 (and) | 1.00 | Solubilizer |
| PEG-40 Hydrogenated | | |
| Castor Oil (and) Water | | |
| Fragrance | 0.20 | Fragrance |
| Biotinoyl Tripeptide-1 | 3.00 | Active |
| containing composition | | |
| Ginseng extract | 0.50 | Promotes |
| | | hair growth |
| Biotin | 0.10 | Active |
| Hydrolyzed wheat protein | 0.5-5.0% | Active |
| complex | | |

Hydrolyzed wheat protein complex is a composition comprising Aqua (and) Glycerin (and) PEG-40 Hydrogenated Castor Oil (and) Hydrolyzed Wheat Protein (and) Caprylyl Butyrate. The lotion is made as described in Example 3, with the addition of hydrolyzed wheat protein complex to the main container at 40° C.

### Example 7

A lotion for treatment of or prevention of hair loss and having CBD is prepared using the ingredients in table 7:

TABLE 7

| | Amount | |
|---|---|---|
| Ingredient | (weight/weight) % | Function |
| Water | Up to 100% | Solvent/Carrier |
| Caffeine | 0.50 | Stimulation of |
| | | hair growth |
| Ethanol | 25.00 | Solvent |
| CBD (Isolate, 97%) | 0.50 | Active |
| | | ingredient |
| Hemp Oil | 0.50 | Carrier |
| Butylene glycol | 2.00 | Humectant |
| Phenoxyethanol 90.0% | 1.00 | Preservative |
| Ethylhexylglycerin 10.0% | | |
| PPG-26-Buteth-26 (and) | 1.00 | Solubilizer |
| PEG-40 Hydrogenated | | |
| Castor Oil (and) Water | | |
| Fragrance | 0.20 | Fragrance |
| Biotinoyl Tripeptide-1 | 3.00 | Active |
| containing composition | | |
| Zinc salt & hydrolyzed soy | 0.5-10 | Active |
| proteins | | |

Zinc salt & hydrolyzed soy proteins is a commercially available compositions comprising: Water (and) Panax ginseng Root Extract (and) Arginine (and) Acetyl Tyrosine (and) Arctium Majus Root Extract (and) Hydrolyzed Soy Protein (and) Polyquaternium-11 (and) PEG-12 Dimethicone (and) Calcium Pantothenate (and) Zinc Gluconate (and) Niacinamide (and) Ornithine HCl (and) Citrulline (and) Glucosamine HCl (and) Biotin. The lotion is made as described in Example 1, with the addition of Zinc salt & hydrolyzed soy proteins to the main container at 40° C.

### Example 8

A lotion for treatment of or prevention of hair loss and having CBD is prepared using the ingredients in table 8:

TABLE 8

| | Amount | |
|---|---|---|
| Ingredient | (weight/weight) % | Function |
| Water | 64.30 | Solvent/Carrier |
| Caffeine | 0.50 | Stimulation of |
| | | hair growth |
| Ethanol | 25.00 | Solvent |
| CBD (Isolate, 97%) | 0.50 | Active |
| | | ingredient |
| Hemp Oil | 0.50 | Carrier |
| Butylene glycol | 2.00 | |
| Phenoxyethanol 90.0% | 1.00 | Preservative |
| Ethylhexylglycerin 10.0% | | |
| PPG-26-Buteth-26 (and) | 1.00 | Solubilizer |
| PEG-40 Hydrogenated | | |
| Castor Oil (and) Water | | |
| Fragrance | 0.20 | Fragrance |
| Biotinoyl Tripeptide-1 | 3.00 | Active |
| containing composition | | |
| Chondrus Crispus Extract | 2.00 | Active |

The lotion is made as described in Example 7, with the modification that Chrondus crispus extract is added in place of zinc salt & hydrolyzed soy proteins.

### Example 9

A lotion for treatment of or prevention of hair loss and having CBD is prepared using the ingredients in table 9:

TABLE 9

| | Amount | |
|---|---|---|
| Ingredient | (weight/weight) % | Function |
| Water | Up to 100% | Solvent/Carrier |
| Caffeine | 0.50 | Stimulation of |
| | | hair growth |
| Ethanol | 25.00 | Solvent |
| CBD (Isolate, 97%) | 0.50 | Active |
| | | ingredient |
| Hemp Oil | 0.50 | Carrier |
| Butylene glycol | 2.00 | Humectant |
| Phenoxyethanol 90.0% | 1.00 | Preservative |
| Ethylhexylglycerin 10.0% | | |
| PPG-26-Buteth-26 (and) | 1.00 | Solubilizer |
| PEG-40 Hydrogenated | | |
| Castor Oil (and) Water | | |
| Fragrance | 0.20 | Fragrance |
| Biotinoyl Tripeptide-1 | 3.00 | Active |
| containing composition | | |
| Ocimum Basilicum Hairy | 0.20-3.00 | Active |
| Root Culture Extract | | |

The lotion is made as described in Example 7, with the modification that Ocimum basilicum Hairy Root Culture Extract available from Mibelle (Buchs, Switzerland) is added in place of zinc salt & hydrolyzed soy proteins.

### Example 10

A lotion for treatment of or prevention of hair loss and having CBD is prepared using the ingredients in table 10:

TABLE 10

| | Amount | |
|---|---|---|
| Ingredient | (weight/weight) % | Function |
| Water | Up to 100% | Solvent/Carrier |
| Caffeine | 0.50 | Stimulation of |
| | | hair growth |
| Ethanol | 25.00 | Solvent |
| CBD (Isolate, 97%) | 0.50 | Active |
| | | ingredient |
| Hemp Oil | 0.50 | Carrier |
| Butylene glycol | 2.00 | Humectant |
| Phenoxyethanol 90.0% | 1.00 | Preservative |
| Ethylhexylglycerin 10.0% | | |
| PPG-26-Buteth-26 (and) | 1.00 | Solubilizer |
| PEG-40 Hydrogenated | | |
| Castor Oil (and) Water | | |
| Fragrance | 0.20 | Fragrance |
| Biotinoyl Tripeptide-1 | 3.00 | Active |
| containing composition | | |
| Pisum Sativum (Pea) Sprout | 0.2-4.0 | Active |
| Extract aqueous solution | | |

Pisum sativum (Pea) Sprout Extract aqueous solution is a product available from Mibelle (Buchs, Switzerland) that comprises Pisum sativum (Pea) Sprout Extract (and) Isomalt (and) Aqua. The lotion is made as described in Example 7, with the modification that Pisum sativum sprout extract is added in place of zinc salt & hydrolyzed soy proteins.

### Example 11

A lotion for treatment of or prevention of hair loss and having CBD is prepared using the ingredients in table 11:

TABLE 11

| | Amount | |
|---|---|---|
| Ingredient | (weight/weight) % | Function |
| Water | 66.20 | Solvent/Carrier |
| Caffeine | 0.5 | Stimulation of |
| | | hair growth |
| Ethanol | 25.00 | Solvent |
| CBD (Isolate, 97%) | 0.50 | Active |
| | | ingredient |
| Hemp Oil | 0.50 | Carrier |
| Butylene glycol | 2.00 | Humectant |
| Phenoxyethanol 90.0% | 1.00 | Preservative |
| Ethylhexylglycerin 10.0% | | |
| PPG-26-Buteth-26 (and) | 1.00 | Solubilizer |
| PEG-40 Hydrogenated | | |
| Castor Oil (and) Water | | |
| Fragrance | 0.20 | Fragrance |
| Biotinoyl Tripeptide-1 | 3.00 | Active |
| containing composition | | |
| Capsicum Frutescens Fruit | 2.00 | Active |
| Extract | | |

The lotion is made as described in Example 7, with the modification that Capsicum frutescens fruit extract is added in place of zinc salt & hydrolyzed soy proteins.

### Example 12

A lotion for treatment of or prevention of hair loss and having CBD is prepared using the ingredients in table 12:

TABLE 12

| | Amount | |
|---|---|---|
| Ingredient | (weight/weight) % | Function |
| Water | 66.01 | Solvent/Carrier |
| Caffeine | 0.50 | Stimulation of |
| | | hair growth |
| Ethanol | 25.00 | Solvent |
| CBD (Isolate, 97%) | 0.50 | Active |
| | | ingredient |
| Hemp Oil | 0.50 | Carrier |
| Butylene glycol | 2.00 | Humectant |
| Phenoxyethanol 90.0% | 1.00 | Preservative |
| Ethylhexylglycerin 10.0% | | |
| PPG-26-Buteth-26 (and) | 1.00 | Solubilizer |
| PEG-40 Hydrogenated | | |
| Castor Oil (and) Water | | |
| Fragrance | 0.20 | Fragrance |
| Biotinoyl Tripeptide-1 | 3.00 | Active |
| containing composition | | |
| Panthenyl Ethyl Ether | 0.10 | Active |
| Inositol | 0.10 | Active |
| Milk Protein | 0.03 | Active |
| Acetyl Cysteine | 0.03 | Active |
| Acetyl Methionine | 0.03 | Active |

The lotion is made as described in Example 7, with the modification that the last 5 ingredients are added in place of zinc salt & hydrolyzed soy proteins.

### Example 13

A lotion for treatment of or prevention of hair loss and having CBD is prepared using the ingredients in table 13:

TABLE 13

| | Amount | |
|---|---|---|
| Ingredient | (weight/weight) % | Function |
| Water | 64.30 | Solvent/Carrier |
| Caffeine | 0.50 | Stimulation of |
| | | hair growth |
| Ethanol | 25.00 | Solvent |
| CBD (Isolate, 97%) | 0.50 | Active |
| | | ingredient |
| Hemp Oil | 0.50 | Carrier |
| Minoxidil | 5.00 | Active |
| | | ingredient |
| Butylene glycol | 2.00 | Humectant |
| Phenoxyethanol 90.0% | 1.00 | Preservative |
| Ethylhexylglycerin 10.0% | | |
| PPG-26-Buteth-26 (and) | 1.00 | Solubilizer |
| PEG-40 Hydrogenated | | |
| Castor Oil (and) Water | | |
| Fragrance | 0.20 | Fragrance |

In a main container, water and caffeine are heated to 75° C. and stirred until dissolved, then cooled while stirring. In another container, hemp oil, CBD and minoxidil are dissolved in ethanol at 45° C. and added to the main container. In another container, butylene glycol is combined with preservative (Phenoxyethanol 90.0%/Ethylhexylglycerin 10.0%) and added to the main container. In another container, solubilizer is mixed with the perfume and added while mixing to the main container.

### Example 14

A lotion for treatment of or prevention of hair loss and having CBD is prepared using the ingredients in table 14:

TABLE 14

| | Amount | |
|---|---|---|
| Ingredient | (weight/weight) % | Function |
| Water | 65.20 | Solvent/Carrier |
| Caffeine | 0.50 | Stimulation of |
| | | hair growth |
| Ethanol | 25.00 | Solvent |
| CBD (Isolate, 97%) | 0.50 | Active |
| | | ingredient |
| Hemp Oil | 0.50 | Carrier |
| Butylene glycol | 2.00 | Humectant |
| Phenoxyethanol 90.0% | 1.00 | Preservative |
| Ethylhexylglycerin 10.0% | | |
| PPG-26-Buteth-26 (and) | 1.00 | Solubilizer |
| PEG-40 Hydrogenated | | |
| Castor Oil (and) Water | | |
| Fragrance | 0.20 | Fragrance |
| Biotinoyl Tripeptide-1 | 3.00 | Active |
| containing composition | | |
| Biotin | 0.10 | Active |
| Diaminopyrimidine Oxide | 1.00 | Active |
| Oxothiazolidinecarboxylate | | |

The lotion is made as described in Example 2, with the addition of biotin and Diaminopyrimidine Oxide Oxothiazolidinecarboxylate in place of the ginseng extract.

### Example 15

A lotion for treatment of or prevention of hair loss and having CBD is prepared using the ingredients in table 15:

TABLE 15

| | Amount | |
|---|---|---|
| Ingredient | (weight/weight) % | Function |
| Water | 64.20 | Solvent/Carrier |
| Caffeine | 0.50 | Stimulation of |
| | | hair growth |
| Ethanol | 25.00 | Solvent |
| CBD (Isolate, 97%) | 0.50 | Active |
| | | ingredient |
| Hemp Oil | 0.50 | Carrier |
| Butylene glycol | 2.00 | Humectant |
| Phenoxyethanol 90.0% | 1.00 | Preservative |
| Ethylhexylglycerin 10.0% | | |
| PPG-26-Buteth-26 (and) | 1.00 | Solubilizer |
| PEG-40 Hydrogenated | | |
| Castor Oil (and) Water | | |
| Fragrance | 0.20 | Fragrance |
| Biotinoyl Tripeptide-1 | 3.00 | Active |
| containing composition | | |
| Biotin | 0.10 | Active |
| Linderane | 0.04 | Active |
| Linderalactone | 0.06 | Active |
| Polyphenols | 0.60 | Active |

The last three ingredients are components of a Lindera strychnifolia root extract.

The lotion is made as described in Example 2, with the addition of the last four ingredients in place of the ginseng extract.

### Example 16

A lotion for treatment of or prevention of hair loss and having CBD is prepared using the ingredients in table 16:

TABLE 16

| | Amount | |
|---|---|---|
| Ingredient | (weight/weight) % | Function |
| Water | 65.50 | Solvent/Carrier |
| Caffeine | 0.50 | Stimulation of |
| | | hair growth |
| Ethanol | 25.00 | Solvent |
| CBD (Isolate, 97%) | 0.50 | Active |
| | | ingredient |
| Hemp Oil | 0.50 | Carrier |
| Bimatoprost | 5.00 | Active |
| | | ingredient |
| Butylene glycol | 2.00 | Humectant |
| Phenoxyethanol 90.0% | 1.00 | Preservative |
| Ethylhexylglycerin 10.0% | | |

In a main container, water and caffeine are heated to 75° C. and stirred until dissolved, then cooled while stirring. In another container, hemp oil, CBD and minoxidil are dissolved in ethanol at 45° C. and added to the main container. In another container, butylene glycol is combined with preservative (Phenoxyethanol 90.0%/Ethylhexylglycerin 10.0%) and added to the main container.

### Example 17

### Determination of the Efficacy of the Composition

An efficacy test is performed to determine the anti-hair loss, hair strengthening, hair growth and hair thickness of the compositions described in Examples 1-16.

Twenty men and ten women volunteers suffering from hair loss, aged between 30 and 55 years old, are enlisted for the study. The study duration is 120 days. Efficacy of the compositions is determined using a dermatological evaluation to verify that skin tolerance and efficacy of the product at time 0, 30 days, 60 days, 90 days and 120 days.

In addition, the state of sebaceous secretion at these time points using high-definition macrophotographs of the scalp. Hair loss and strength is also determined by counting hair shed at washing and styling at these timepoint. Topographic imaging using the Visioscan® is used to determine hair condition to measure growth and thickness parameters. The patients subjectively analyze the compositions used, by filling a questionnaire at the beginning of the test (which includes aspects related to their consumption habits of this type of product). At the end of the test, the volunteers fill out a questionnaire with information about the organoleptic perception of the product, the efficacy of the product and the future use of the product.

It is suggested that compositions testing comprising CBD are effective in renewing hair growth and improving hair strength. It is further suggested that compositions tested comprising CBD and additional agents act synergistically to stimulate hair growth and improve hair strength.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method for treatment of a patient suffering from hair loss comprising administering to the patient a composition comprising a cannabinoid, applied via the topical route to the skin or scalp of a patient in need thereof.

2. The method according to claim 1 wherein the composition further comprises at least one additional active agent.

3. The method according to claim 1 wherein the at least one additional active agent is selected from the group consisting of: caffeine, minoxidil, bimatoprost, ginseng extract, Biotinoyl Tripeptide-1, Myristoyl Pentapeptide-17, Octapeptide-2, hydrolyzed wheat protein, arginine, Arctium majus Root Extract, Chondrus crispus Extract, Ocimum basilicum Hairy Root Culture Extract, Pisum sativum (Pea) Sprout Extract, Capsicum frutescens Fruit Extract, Milk protein, Diaminopyrimidine Oxide Oxothiazolidinecarboxylate, apigenin, oleanoic acid and Lindera strychnifolia Root Extract.

4. The method according to claim 1 wherein the composition is in the form of an emulsion, gel, cream, lotion, spray, eye liner, mascara, hair oil, or hair serum.

5. The method according to claim 1 wherein the cannabinoid is present in the composition in an amount between 0.001% and 5% by weight.

6. The method according to any one of the previous claims wherein the cannabinoid is CBD.

7. The method according to claim 6 wherein the CBD is present in an amount of 0.5% by weight of the composition.

8. The method according to claim 1 wherein the composition is left on the skin or scalp of the patient for at least one hour.

9. The method according to claim 1 wherein the composition is applied in combination with microneedling.

10. The method according to claim 1 wherein the amount administered of the composition per administration is between 0.1 g and 30 g.

11. The method according to claim 2 wherein the additional active agent is present in the composition in an amount of between 0.0001% by weight and 5% by weight.

12. The method according to claim 2 wherein an additional agent is caffeine.

13.-24. (canceled)

25. The method according to claim 1 wherein the composition is in the form of a hair serum.


SEQUENCE LISTING (2 sequences)

SEQ ID NO: 1 - protein, 5 aa, synthetic construct
  LIPID 1: note = N-myristoyl lysine
KLAKK

SEQ ID NO: 2 - protein, 5 aa, synthetic construct
EQLKT